(19) United States
(12) Patent Application Publication
(10) Pub. No.: US 2007/0173872 A1
(43) Pub. Date: Jul. 26, 2007

(54) Surgical instrument for cutting and coagulating patient tissue

(75) Inventor: Steven K. Neuenfeldt, Pleasanton, CA (US)

Correspondence Address: Thompson Hine LLP, 2000 Courthouse Plaza N.E., 10 West Second Street, Dayton, OH, 45402-1758, US

(73) Assignee: Ethicon Endo-Surgery, Inc.

(21) Appl. No.: 11/337,405

(22) Filed: Jan. 23, 2006

(51) Int. Cl. A61B 17/32

(52) U.S. Cl. 606/169

(57) ABSTRACT

A first medical instrument is for cutting and coagulating patient tissue and includes a two-pronged end effector. The end effector has a first prong which includes a medical ultrasonic blade, has a second prong which includes an arm opposing the ultrasonic blade, and has a surgical knife. A second medical instrument is for a medical instrument for cutting and coagulating patient tissue and includes a two-pronged end effector. The end effector has a first prong which includes a first bipolar radio-frequency electrode, has a second prong which includes an arm opposing the electrodes, and has a surgical knife.

### FIELD OF THE INVENTION

The present invention is related generally to surgical instruments, and more particularly to a surgical instrument for cutting and coagulating patient tissue.

### BACKGROUND OF THE INVENTION

Ultrasonic surgical instruments are known which include an end effector which is an ultrasonic surgical shears having an ultrasonic surgical blade, a clamping arm operable to open and close toward the blade, a tissue pad attached to the clamping arm and including a clamping surface area, and a device for exerting a clamping force on the clamping arm which creates a clamping pressure on a blood vessel which is positioned between the clamping surface area of the tissue pad and the blade. It is noted that the clamping surface area is the area where the blade and the tissue pad are in close proximity when the clamping arm is in a closed position. Exemplary devices are described in U.S. Pat. Nos. 5,322,055 and 6,325,811, the contents of which are incorporated herein by reference. The result of the ultrasonically-vibrating ultrasonic surgical blade and the clamping pressure on the blood vessel is a coaptation of the blood vessel (a bringing together of the walls of the blood vessel), a transection (a cutting) of the coaptated blood vessel, and a coagulation (a sealing) of the coaptated cut ends of the blood vessel.

A surgical instrument is known which has an end effector which includes a pair of radio-frequency bipolar electrodes which are used to transect and coagulate patient tissue. Surgical knifes (i.e., surgical instruments having a sharp cutting edge) are known for transecting patient tissue.

Still, scientists and engineers continue to seek improved surgical instruments for cutting and coagulating patient tissue.

### SUMMARY OF THE INVENTION

A first expression of a first embodiment of the invention is for a medical instrument for cutting and coagulating patient tissue. The medical instrument includes a two-pronged end effector having a first prong which includes a medical ultrasonic blade, having a second prong which includes an arm opposing the ultrasonic blade, and having a surgical knife.

A second expression of a first embodiment of the invention is for a medical instrument for cutting and coagulating patient tissue. The medical instrument includes a two-pronged end effector having a first prong which includes a medical ultrasonic blade, having a second prong which includes an arm opposing the ultrasonic blade, and having a surgical knife. The ultrasonic blade is an ultrasonic blade portion of an ultrasonic surgical shears, and the arm is a clamp arm portion of the ultrasonic surgical shears. The surgical knife is attached to the ultrasonic blade. The surgical blade has at least one vibration node, and the surgical knife is spaced apart from each of the at-least-one vibration node.

A first expression of a second embodiment of the invention is for a medical instrument for cutting and coagulating patient tissue. The medical instrument includes a two-pronged end effector having a first prong which includes a first bipolar radio-frequency electrode, having a second prong which includes an arm opposing the first bipolar radio-frequency electrode, and having a surgical knife.

Several benefits and advantages are obtained from one or more of the expressions of embodiments of the invention. In one example, the surgical knife is used to cut avascular patient tissue (such as, but not limited to, avascular mesentery tissue). In the same or a different example, the ultrasound blade or the radio-frequency electrode is used to cut and coagulate vascular patient tissue (such as, but not limited to, vascular mesentery tissue). In one variation, the surgical knife vibrates with the ultrasound blade for faster cutting of patient tissue, and the surgical knife is rotatable to avoid patient tissue being cut by the ultrasound blade. In the same or a different variation, the medical instrument places patient tissue in tension between the two prongs for faster cutting.

The present invention has, without limitation, application in hand-activated instruments as well as in robotic-assisted instruments. The medical ultrasonic blade embodiment of the invention has, without limitation, application with straight or curved ultrasonic surgical blades as disclosed in the patents incorporated by reference.

### BRIEF DESCRIPTION OF THE FIGURES

FIG. 1 is a schematic, side-elevational view of a first embodiment of the invention showing a medical instrument including a medical ultrasonic blade, an arm, and a surgical knife;

FIG. 2 is a schematic, side-elevational view of a second embodiment of the invention showing a medical instrument including a pair of bipolar radio-frequency electrodes (only one of which is shown in FIG. 2), an arm, and a surgical knife; and

FIG. 3 is a view taken along lines 3-3 in FIG. 2 showing both electrodes.

### DETAILED DESCRIPTION OF THE INVENTION

Before explaining the present invention in detail, it should be noted that the invention is not limited in its application or use to the details of construction and arrangement of parts illustrated in the accompanying drawings and description. The illustrative embodiments of the invention may be implemented or incorporated in other embodiments, variations and modifications, and may be practiced or carried out in various ways. Furthermore, unless otherwise indicated, the terms and expressions employed herein have been chosen for the purpose of describing the illustrative embodiments of the present invention for the convenience of the reader and are not for the purpose of limiting the invention.

It is understood that any one or more of the following-described embodiments, examples, etc. can be combined with any one or more of the other following-described embodiments, examples, etc.

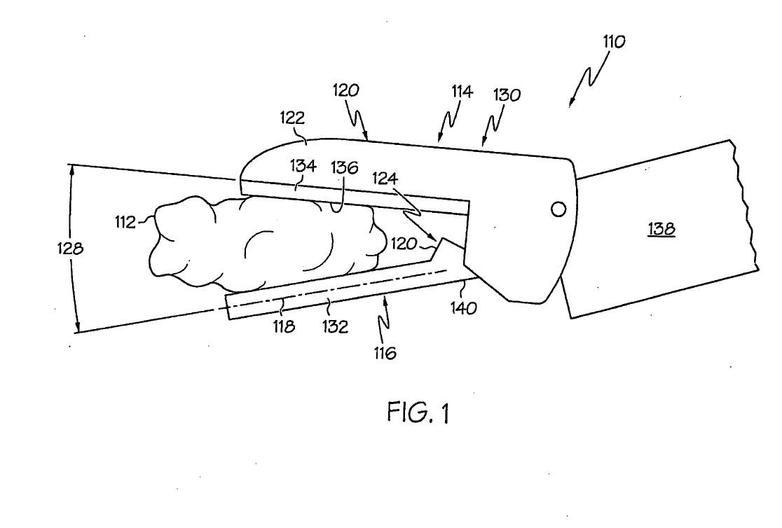

A first embodiment of the invention is shown in FIG. 1. A first expression of the embodiment of FIG. 1 is for a medical instrument 110 for cutting and coagulating patient tissue 112. The medical instrument 110 comprises a two-pronged (i.e., at least two-pronged) end effector 114 having a first prong 116 which includes a medical ultrasonic blade 118, having a second prong 120 which includes an arm 122 opposing the ultrasonic blade 118, and having a surgical knife 124.

It is noted that a medical ultrasonic blade 118 does not include a sharp cutting edge but relies on ultrasonic vibration to transect patient tissue 112 as is known to those skilled in the art. It is also noted that a surgical knife 120 includes a sharp cutting edge 126 adapted for transecting patient tissue 112.

In one enablement of the first expression of the embodiment of FIG. 1, the surgical knife 124 is attached to one of the ultrasonic blade 118 and the arm 122. In one variation, the surgical knife 124 is attached (such as, without limitation, monolithically attached or mechanically and/or adhesively attached) to the ultrasonic blade 118. In another variation, not shown, the surgical knife is attached (such as, without limitation, monolithically attached or mechanically and/or adhesively attached) to the arm. In one extension, not shown, a second surgical knife is attached to the other of the ultrasonic blade and the arm. In the same or a different extension, at least two surgical knives are attached to the ultrasonic blade and/or the arm.

In one configuration of the first expression of the embodiment of FIG. 1, the ultrasonic blade 118 is disposed at an angle 128 with respect to the arm 122. In one variation, the surgical knife 124 is always angularly spaced apart from the other of the ultrasonic blade 118 and the arm 122. In a first modification of this variation, the angle 128 is invariant. In a second modification of this variation, the angle 128 is a user-adjustable angle. In a first example of this second modification, the ultrasonic blade 118 and the arm 122 at least partially define an ultrasonic surgical shears 130, wherein the angle 128 is user-decreased (down to a minimum angle which still keeps the surgical knife 124 always spaced apart from the other of the ultrasonic blade 118 and the arm 122) to grasp patient tissue 112 between the ultrasonic blade 118 and the arm 122, and wherein the angle 128 is user-increased to release the grasped patient tissue. Mechanisms (which may include pivots, cables, and hand levers with stops) to accomplish such movement and such minimum angle are within the ordinary level of skill of designers of ultrasonic surgical shears. In a second example of this second modification, the ultrasonic blade 118 and the arm 122 are devoid of any patient tissue clamping and unclamping mode of operation (but still allow the user to adjust and lock the adjusted angle, wherein mechanisms [which may include knobs, cables, and pivots] to accomplish such adjusting and such locking are within the level of skill of designers of ultrasonic surgical shears).

A second expression of the embodiment of FIG. 1 is for a medical instrument 110 for cutting and coagulating patient tissue 112. The medical instrument 110 comprises a two-pronged (i.e., at least two-pronged) end effector 114 having a first prong 116 which includes a medical ultrasonic blade 118, having a second prong 120 which includes an arm 122 opposing the ultrasonic blade 118, and having a surgical knife 124. The ultrasonic blade 118 is an ultrasonic blade portion of an ultrasonic surgical shears 130, and the arm 122 is a clamp arm portion of the ultrasonic surgical shears 130. The surgical knife 124 is attached to the ultrasonic blade 118. The ultrasonic blade 118 has at least one vibration node, and the surgical knife 124 is spaced apart from each of the at-least-one vibration node.

For a blade undergoing a longitudinal component of vibration, a longitudinal vibration node is a location on the blade which experiences no longitudinal vibration as is known to the artisan. Likewise, for a blade undergoing a transverse (i.e., bending) component of vibration, a transverse vibration node is a location on the blade which experiences no transverse vibration, and for a blade undergoing a torsional (i.e., twisting) component of vibration, a torsional vibration node is a location on the blade which experiences no torsional vibration.

In one enablement of the second expression of the embodiment of FIG. 1, the ultrasonic blade 118 has a longitudinal axis 132, and the ultrasonic blade 118 is rotatable about the longitudinal axis 132 without rotating the arm 122. Mechanisms (which may include motors) for such rotation are within the ordinary level of skill of designers of medical end effectors. In one construction of the second expression of the embodiment of FIG. 1, the end effector 114 includes a tissue pad 134 which is attached to the arm 122 and which includes a clamping surface area 136.

In one implementation of the second expression of the embodiment of FIG. 1, the medical instrument 110 includes a sheath 138 operatively supporting the end effector 114, wherein, although not shown, a waveguide portion of the ultrasonic blade 118 extends within the sheath 138 and is operatively connected to an ultrasonic transducer housed in a handpiece. In one deployment of the second expression of the embodiment of FIG. 1, the first prong 116 and the arm 122 define a pair of jaws, wherein the surgical knife 124 is disposed proximate the proximal end of the jaw defined by the first prong 116. In one illustration of the second expression of the embodiment of FIG. 1, the ultrasonic surgical shears 130 is used, without limitation, for vessel sealing, tissue grasping, tissue dissecting, tissue backcutting, and tissue spot sealing.

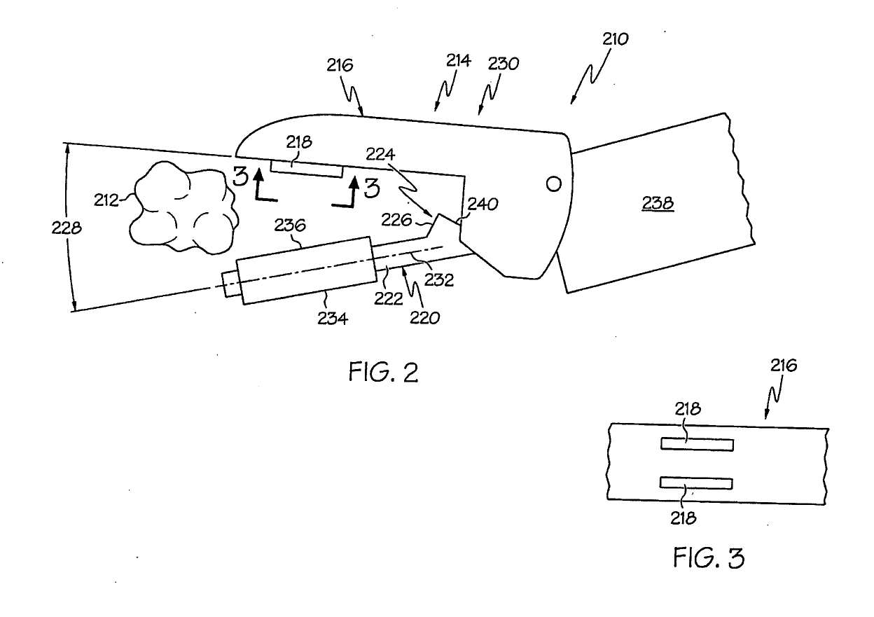

A second embodiment of the invention is shown in FIG. 2. A first expression of the embodiment of FIG. 2 is for a medical instrument 210 for cutting and coagulating patient tissue 212. The medical instrument 210 comprises a two-pronged (i.e., at least two-pronged) end effector 214 having a first prong 216 which includes a first bipolar radio-frequency electrode (the upper one of the electrodes 218 of FIG. 3), having a second prong 220 which includes an arm 222 opposing the electrodes 218, and having a surgical knife 224. In one example, one of the first and second prongs 216 and 220 includes a second bipolar radio-frequency electrode (the lower one of the electrodes 218 of FIG. 3 which shows an example of the first prong 216 as having both electrodes 218). Other examples, including those having additional electrodes, are left to those skilled in the art.

It is noted that the pair of bipolar radio-frequency electrodes 218 do not include a sharp cutting edge but rely on resistive heating to transect patient tissue 212 as is known to those skilled in the art. It is also noted that a surgical knife 220 includes a sharp cutting edge 226 adapted for transecting patient tissue 212.

In one enablement of the first expression of the embodiment of FIG. 2, the surgical knife 224 is attached to one of the first prong 216 and the arm 222. In one variation, the surgical knife 224 is attached (such as, without limitation, monolithically attached or mechanically and/or adhesively attached) to the arm 222. In another variation, not shown, the surgical knife is attached (such as, without limitation, monolithically attached or mechanically and/or adhesively attached) to the first prong. In one extension, not shown, a second surgical knife is attached to the other of the first prong and the arm. In the same or a different extension, at least two surgical knives are attached to the first prong and/or the arm.

In one configuration of the first expression of the embodiment of FIG. 2, the first prong 216 is disposed at an angle 228 with respect to the arm 222. In one variation, the surgical knife 224 is always angularly spaced apart from the other of the first prong 216 and the arm 222. In a first modification of this variation, the angle 228 is invariant. In a second modification of this variation, the angle 228 is a user-adjustable angle. In a first example of this second modification, the first prong 216 and the arm 222 at least partially define a radio-frequency surgical shears 230, wherein the angle 228 is user-decreased (down to a minimum angle which still keeps the surgical knife 224 always spaced apart from the other of the first prong 216 and the arm 222) to grasp patient tissue 212 between the first prong 216 and the arm 222, and wherein the angle 228 is user-increased to release the grasped patient tissue. Mechanisms (which may include pivots, cables, and hand levers with stops) to accomplish such movement and such minimum angle are within the ordinary level of skill of designers of ultrasonic surgical shears. In a second example of this second modification, the first prong 216 and the arm 222 are devoid of any patient tissue clamping and unclamping mode of operation (but still allow the user to adjust and lock the adjusted angle, wherein mechanisms [which may include knobs, cables, and pivots] to accomplish such adjusting and such locking are within the level of skill of designers of ultrasonic surgical shears).

In one arrangement of the first expression of the embodiment of FIG. 2, the first prong 216 is an electrode-supporting portion of a radio-frequency surgical shears 230, and the arm 222 is a clamp arm portion of the radio-frequency surgical shears 230. In this arrangement, the surgical knife 124 is attached to the arm 222.

In one enablement of the first expression of the embodiment of FIG. 2, the arm 222 has a longitudinal axis 232, and the arm 218 is rotatable about the longitudinal axis 232 without rotating the first prong 216. Mechanisms (which may include motors) for such rotation are within the ordinary level of skill of designers of medical end effectors. In one construction of the first expression of the embodiment of FIG. 2, the end effector 214 includes a tissue pad 234 which is attached to the arm 222 and which includes a clamping surface area 236.

In one implementation of the first expression of the embodiment of FIG. 2, the medical instrument 210 includes a sheath 238 operatively supporting the end effector 214 In one deployment of the first expression of the embodiment of FIG. 2, the first prong 216 and the second prong 220 define a pair of jaws, wherein the surgical knife 224 is disposed proximate the proximal end of the jaw defined by the second prong 220. In one illustration of the first expression of the embodiment of FIG. 2, the radio-frequency surgical shears 230 is used, without limitation, for vessel sealing, tissue grasping, tissue dissecting, tissue backcutting, and tissue spot sealing.

Several benefits and advantages are obtained from one or more of the expressions of embodiments of the invention. In one example, the surgical knife is used to cut avascular patient tissue (such as, but not limited to, avascular mesentery tissue). In the same or a different example, the ultrasound blade or the radio-frequency electrode is used to cut and coagulate vascular patient tissue (such as, but not limited to, vascular mesentery tissue). In one variation, the surgical knife vibrates with the ultrasound blade for faster cutting of patient tissue, and the surgical knife is rotatable to avoid patient tissue being cut by the ultrasound blade. In the same or a different variation, the medical instrument places patient tissue in tension between the two prongs for faster cutting.

While the present invention has been illustrated by a description of several embodiments, it is not the intention of the applicant to restrict or limit the spirit and scope of the appended claims to such detail. Numerous other variations, changes, and substitutions will occur to those skilled in the art without departing from the scope of the invention. For instance, the medical instrument embodiments of the invention have application in robotic assisted surgery taking into account the obvious modifications of such systems, components to be compatible with such a robotic system. It will be understood that the foregoing description is provided by way of example, and that other modifications may occur to those skilled in the art without departing from the scope and spirit of the appended Claims.

What is claimed is:

1. A medical instrument for cutting and coagulating patient tissue comprising a two-pronged end effector having a first prong which includes a medical ultrasonic blade, having a second prong which includes an arm opposing the ultrasonic blade, and having a surgical knife.

2. The medical instrument of claim 1, wherein the surgical knife is attached to one of the ultrasonic blade and the arm.

3. The medical instrument of claim 2, wherein the ultrasonic blade is disposed at an angle with respect to the arm.

4. The medical instrument of claim 3, wherein the surgical knife is always angularly spaced apart from the other of the ultrasonic blade and the arm.

5. The medical instrument of claim 4, wherein the angle is invariant.

6. The medical instrument of claim 4, wherein the angle is a user-adjustable angle.

7. The medical instrument of claim 6, wherein the ultrasonic blade and the arm at least partially define an ultrasonic surgical shears, wherein the angle is user-decreased to grasp patient tissue between the ultrasonic blade and the arm, and wherein the angle is user-increased to release the grasped patient tissue.

8. The medical instrument of claim 6, wherein the ultrasonic blade and the arm are devoid of any patient tissue clamping and unclamping mode of operation.

9. A medical instrument for cutting and coagulating patient tissue comprising a two-pronged end effector having a first prong which includes a medical ultrasonic blade, having a second prong which includes an arm opposing the ultrasonic blade, and having a surgical knife, wherein the medical ultrasonic blade is an ultrasonic blade portion of an ultrasonic surgical shears, wherein the arm is a clamp arm portion of the ultrasonic surgical shears, wherein the surgical knife is attached to the ultrasonic blade, wherein the surgical blade has at least one vibration node, and wherein the surgical knife is spaced apart from each of the at-least-one vibration node.

10. The medical instrument of claim 1, wherein the ultrasonic blade has a longitudinal axis, and wherein the ultrasonic blade is rotatable about the longitudinal axis without rotating the arm.

11. A medical instrument for cutting and coagulating patient tissue comprising a two-pronged end effector having a first prong which includes a first bipolar radio-frequency electrode, having a second prong which includes an arm opposing the electrodes, and having a surgical knife.

12. The medical instrument of claim 11, wherein the surgical knife is attached to one of the first prong and the arm.

13. The medical instrument of claim 12, wherein the first prong is disposed at an angle with respect to the arm.

14. The medical instrument of claim 13, wherein the surgical knife is always angularly spaced apart from the other of the first prong and the arm.

15. The medical instrument of claim 14, wherein the angle is invariant.

16. The medical instrument of claim 14, wherein the angle is a user-adjustable angle.

17. The medical instrument of claim 16, wherein the first prong and the arm at least partially define a radio-frequency surgical shears, wherein the angle is user-decreased to grasp patient tissue between the first prong and the arm, and wherein the angle is user-increased to release the grasped patient tissue.

18. The medical instrument of claim 16, wherein the first prong and the arm are devoid of any patient tissue clamping and unclamping mode of operation.

19. The medical instrument of claim 11, wherein the first prong is an electrode-supporting portion of a radio-frequency surgical shears, wherein the arm is a clamp arm portion of the radio-frequency surgical shears, and wherein the surgical knife is attached to the arm.

20. The medical instrument of claim 11, wherein the arm has a longitudinal axis, and wherein the arm is rotatable about the longitudinal axis without rotating the first prong.

21. The medical instrument of claim 11, wherein one of the first and second prongs includes a second bipolar radio-frequency electrode.
